(19) United States
(12) Patent Application Publication
(10) Pub. No.: US 2023/0364180 A1
(43) Pub. Date: Nov. 16, 2023

(54) COMPOSITION FOR IMPROVING MILD COGNITIVE IMPAIRMENT AND METHOD FOR IMPROVING MILD COGNITIVE IMPAIRMENT

(71) Applicant: TOKAI BUSSAN CO., LTD., Tokyo (JP)

(72) Inventors: Tatsuhiro HISATSUNE, Tokyo (JP); Nobutaka MASUOKA, Tokyo (JP); Nobuya YANAI, Tokyo (JP); Kenichiro SATO, Tokyo (JP); Shigenobu SHIOTANI, Tokyo (JP)

(21) Appl. No.: 18/360,773

(22) Filed: Jul. 27, 2023

Related U.S. Application Data
(62) Division of application No. 16/931,979, filed on Jul. 17, 2020.

(30) Foreign Application Priority Data
Jul. 17, 2019 (JP) 2019-132196

(51) Int. Cl. A61K 38/05; A61P 25/28; A61K 47/12; A61K 47/22

(52) U.S. Cl. CPC A61K 38/05; A61P 25/28; A61K 47/12; A61K 47/22

(57) ABSTRACT

A method for improving mild cognitive impairment of a human patient includes providing a dose of 250 mg-500 mg of a composition once or twice a day for three months or more. The human patient has mild cognitive impairment with a score of 23 points or more and less than 27 points in the Mini-Mental State Examination, a score of 0.5 in the Clinical Dementia Rating, or a score of less than 25 points in the Montreal Cognitive Assessment. The composition includes imidazole dipeptide separated and purified from chicken extract or salmon extract. The imidazole dipeptide does not contain creatinine, and 75% or more of the imidazole dipeptide in the composition is anserine.

### CROSS-REFERENCE TO RELATED APPLICATION

This application is a divisional application of U.S. application Ser. No. 16/931,979, filed Jul. 17, 2020, which claims priority to JP Application No. 2019-132196, filed Jul. 17, 2019, the contents of which are hereby incorporated herein by reference.

### BACKGROUND

### Technical Field

The present invention relates to a composition such as a functional food containing imidazole dipeptide as a main component for improving mild cognitive impairment and a method for improving mild cognitive impairment using the composition.

### Background

The increased number of patients with dementia in conjunction with an aging population has become a serious problem worldwide. The cognitive function of healthy people declines with age, and one in four elderly people may develop dementia.

Dementia (hereinafter DM) is affecting an increasing number of patients in conjunction with an aging population, and is becoming a serious problem not only in Japan but also globally. Although the cognitive function of the brain deteriorates with age, in many cases the deterioration does not become a major impediment to daily life. However, DM is a serious disease that is caused by irreversible degeneration of nerve cells due to a disorder, such as genetic factors and accumulation of amyloid β, and seriously hinders daily life.

Some data may show that 60% of the causes of dementia are Alzheimer's disease (hereinafter AD); however, it is known that in developing dementia, including Alzheimer's disease, a person goes through a phase called mild cognitive impairment (abbreviated as MCI). MCI does not necessarily occur in healthy elderly people, but it is also seen in relatively young and middle-aged people, and half of MCI cases can progress to DM, including Alzheimer's dementia.

Both DM and MCI are accompanied by degeneration of nerve cells/tissues, but the former is irreversible degeneration while the latter is a partially reversible degeneration.

Imidazole dipeptide (abbreviated as IMDP) is a general term for dipeptides composed of β-alanine and L-histidine, and four types thereof are known, carnosine, anserine, balenine, and homocarnosine.

Although the physiological function of IMDP has not been completely elucidated, studies on its neuroprotective action as well as anti-fatigue and anti-oxidant actions have been extensively conducted so far. In particular, studies regarding prevention and treatment of DM have been conducted using an animal model of AD, which is believed to be caused by aggregation and accumulation of amyloid β. In addition, human trials on memory-improving effects have been conducted on healthy people and healthy elderly people.

For example, expected effects of IMDP reported in non-patent literature include amyloid β aggregation inhibitory action in AD onset prevention (1), prevention of oxidative damage of neuronal mitochondria in AD-induced transgenic mice (2), and an effect of improving the survival number of cerebrovascular pericytes (3), and memory improvement by means of increasing cerebral blood volume (4) has been reported in human trials conducted on healthy subjects. In addition, a number of findings have been published in the patent literature, such as on learning ability improving action (1), memory and psychological function improving action (2), and prevention of brain barrier pericyte degenerative disease (3).

The above contents are described in WO2007-116987, Japanese Laid Open Patent Application No. 2015-193582, and Japanese Laid Open Patent Application No. 2018-140958 and Aloisi A. et al., PloS ONE 2013 Jul. 3; 8 (7): e68159, Carlo C. et al., PloS ONE 2011 Mar. 6; (3): e17971, Kaneko J et al. Scientific Rep 7: 12571 DOI: 10.1038/s41598-017-12785-7, Hisatsune T et al., J. Alzheimer Dis. 33 (2013) 983-997, and Eshkoor S A et al., Clinical Interventions in Aging 2015: 10 687-693 discussed below.

### SUMMARY

It has been found that there has not been any method to suppress and provide recovery from the progression from mild cognitive impairment to dementia. The present invention relates to a composition containing a high-purity imidazole dipeptide as an active ingredient, which suppresses the progression from mild cognitive impairment to dementia.

It has been considered impossible to recover damaged nerve cells/tissues at the stage of dementia diagnosis, and progression and deterioration of the disease state can only be insufficiently suppressed, so to date there have not been any effective therapeutic means.

Just as there is no effective treatment for DM, no method has been known thus far to suppress the progression from MCI to DM or to restore it to a normal state.

Imidazol dipeptide (IMDP) is present in the brain and skeletal muscles of vertebrates and is extracted from the meat of livestock or fish and used as a functional health food. However, in addition to IMDP, many impurities such as proteins and amino acids are contained in extracts of natural materials, and caution is required when ingested by elderly people with impaired renal function.

Particularly problematic are protein and potassium, as well as creatinine, which is present in relatively large amounts in animal extracts. Creatinine is an indicator substance for monitoring renal function in diabetes, etc., and the creatinine clearance ability in the kidney is generally reduced in elderly people. Therefore, when a large amount of IMDP is taken as an animal extract by an elderly person, blood creatinine concentration increases, making it difficult to distinguish it from diabetic renal function decline and causing anxiety in the elderly.

The findings of the prior art documents suggest the possibility of DM prevention in healthy people, and the effects in AD model animals indicate the effects in the DM-developed state. The time required for a healthy person to actually develop DM including AD etc. can be between ten years or so to a few decades, and although a certain preventive effect is expected if a healthy person continues to take IMDP, the cost-effectiveness remains extremely unclear in a situation in which the need cannot be assessed.

In addition, even though IMDP is expected to inhibit amyloid-β aggregation and prevent mitochondrial oxidative injury in DM patients with degeneration of nerve cells, because such degeneration is irreversible, it would not treat or improve DM, even though it may slow the progression or worsening of DM, as conventional treatments do, and there is no precedent reported that IMDP is effective in treating DM.

On the other hand, in the process of developing DM in healthy people, there is a period of mild cognitive impairment (MCI), which may be called an earlier stage of DM, and MCI is thought to be a precursor of AD due to amyloid-β aggregation, as described in the prior art literature, degeneration of nerve cells associated with hypertension, diabetes, and dyslipidemia, as well as the damaging effects of genetic factors on nerve cells. In addition, half of those with MCI will likely progress to DM including AD within 5 years.

Therefore, the subjects for whom IMDP should be used to suppress an increase in the number of DM patients and as a prophylactic of DM onset are those with this MCI. However, there have been no findings to date testing the effects of IMPD on MCI.

In addition, excluding chemically synthesized products, IMDP is generally ingested as part of animal meat or fish meat, or extracted from animal materials, but animal extracts contain a large amount of creatinine derived from the living body, and there are problems with the long-term ingestion thereof by elderly people with MCI.

A decrease in renal function is generally observed more with age or with diabetes, which is monitored through blood creatinine levels. When renal function declines, creatinine clearance deteriorates, so if the coexisting creatinine content in IMDP is high, it may become difficult to distinguish whether the creatinine concentration in the blood is elevated due to the decline in renal function, or may cause anxiety regarding IMDP intake in elderly people ingesting IMDP.

In order to more effectively prevent the onset of DM, it was specified that the subjects ingesting IMDP would be middle-aged and elderly people in the MCI state, and that the IMDP used would not contain any biologically derived components such as creatinine, in order to test the effects thereof on preventing progression from MCI to DM as well as on recovering from MCI to normal.

The composition according to embodiments of the present invention contains imidazole dipeptide as a main component, and the imidazole dipeptide contains anserine extracted from chicken meat extract or salmon meat extract and does not contain any creatinine. Furthermore, since IMDP has an antioxidant ability and is susceptible to oxidation in vivo, two or more in vivo antioxidants for IMDP were used, and selected from vitamin C, ferulic acid, vitamin E, and astaxanthin.

The IMDP ingestion subjects in the method for improving MCI embodiments of the present invention are defined as people judged to have MCI by the Mini-Mental State Examination (MMSE) or Clinical Dementia Rating (CDR), which are generally used as cognitive function tests for DM diagnosis. That is, those with an MMSE score of 23 points or more and less than 27 points and a CDR score of 0.1 or more and less than 0.5 were considered. In addition, those who scored 25 or less in the Montreal Cognitive Assessment (abbreviated as MoCA), which has a short test time, were also considered MCI.

The intake of IMDP was set to 500 mg per day, which was ingested once a day, or divided in half and ingested twice a day. The intake period was set to 3 months or more.

The composition for improving mild cognitive impairment according to embodiments of the present invention, which contains IMDP as a main component, can suppress the progression from MCI to DM, improve MCI, and restore a normal state.

### BRIEF DESCRIPTION OF THE DRAWINGS

The features described in this disclosure are set forth with particularity in the appended claims.

FIG. 1A is a chicken extract raw material, in which Vo indicates a high-molecular-weight protein fraction in the column void volume, IMDP indicates a peak of the imidazole dipeptide according to the present invention, and Cre indicates a creatinine peak;

FIG. 1B is a GPC-HPLC chromatogram of IMDP purified from chicken extract;

FIG. 1C is a salmon extract raw material;

FIG. 1D is a GPC-HPLC chromatogram of IMDP (anserine) purified from salmon extract;

FIG. 2A is a hydroxyl radical, in which 1 is untreated target protein, 2 is no antioxidant added, 3 is IMDP added, 4 is vitamin C added, 5 is ferulic acid added, 6 is IMDP+vitamin C+ferulic acid added; and

FIG. 2B is a proteolytic action by a peroxynitrite radical.

### DETAILED DESCRIPTION

### (1) IMDP Preparation

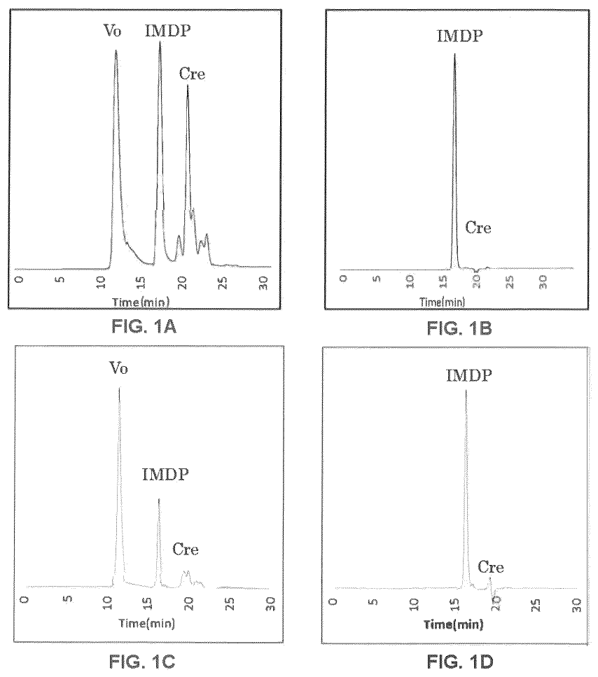

As shown in FIGS. 1A-D, the IMDP preparation relating to the present invention is obtained by subjecting a chicken extract or a salmon meat extract to a concentrated desalting treatment by ion exchange chromatograph and a nanofiltration membrane, to remove proteins, amino acids, creatinine, potassium salts, and the like. For example, the method of Japanese Patent No. 5142126 is used as the manufacturing method.

As for the components in IMDP, the content ratio of anserine:carnosine in chicken extract was 3:1 (75% of anserine), and salmon extract was entirely anserine (100% of anserine). Little protein, creatinine, other amino acids, and potassium salts were contained therein. As an IMDP containing no biological component such as creatinine, for example, the one produced by the enzymatic synthesis method disclosed in JP-A-2018-102287 can be used.

### (2) IMDP Compounding Agent

IMDP shows strong antioxidative activity against hypochlorite radicals among the three types of reactive oxygen species produced in vivo (hypochlorite radicals, hydroxyl radicals, and peroxynitrite radicals). However, IMDP has an extremely weak antioxidant effect on hydroxyl radicals and peroxynitrite radicals.

On the other hand, vitamin E and astaxanthin are known to have strong antioxidant activity against hydroxyl radicals, and vitamin C has strong antioxidant activity against peroxynitrite radicals. Utilizing this fact, in order to prevent IMDP from being easily oxidized by active oxygen in vivo and to fully exert the effect thereof, granules were prepared by adding 25 mg-90 mg of vitamin C per 250 mg of IMDP, and 1-20 mg of ferulic acid and astaxanthin (10-40 mg in the case of vitamin E).

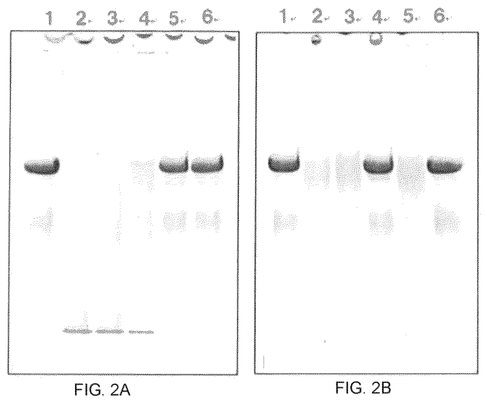

As shown in FIGS. 2A and 2B, ferulic acid has a strong antioxidant activity against hydroxyl radicals and vitamin C has a strong antioxidant activity against peroxynitrite radicals, thus preventing the degradation of target proteins, which would be degraded by these active oxygen species if IMDP was used alone. Therefore, by coexisting with these antioxidants, the antioxidant effect of IMDP is retained both for hydroxyl radicals and peroxynitrite radicals.

### (3) MCI Subject Selection

From the viewpoint of reducing burden on the subjects, primary screening was performed in middle-aged and older persons aged 65 and over using MoCA, which has a relatively short test time. The MoCA score is 25 or less, the MMSE is 23 or more and less than 27, or the CDR score is 0.5. In CDR, a score of 0 is normal, and a score of 1 or more means dementia.

### (4) IMDP Intake Trial

A double-blind, placebo-controlled trial was conducted in 29 middle-aged and elderly people who were determined to be at high risk for MCI. 14 subjects were given 250 mg IMDP purified from salmon extract and IMDP granules containing 15 mg of ferulic acid and 75 mg of vitamin C as antioxidants twice a day in the morning and evening (test diet group). 15 subjects in the placebo diet group also received a placebo diet containing 250 mg of dextran and an antioxidant instead of IMDP twice a day in the morning and evening. After ingestion for 3 months, MCI status was evaluated by means of MoCA, MMSE, or CDR. As shown in the results in Table 1, there was no significant difference in MoCA scores, but a significant improvement effect was seen in the MMSE score. Two patients in the placebo diet group, whose MMSE decreased to 23 or less, were suspected of having clinical dementia (CD) but no such subjects were found in the test diet group.

TABLE 1
MCI improvement effect by IMDP ingestion

| | Before Ingestion | | After ingestion for 3 months | | Improvement Score | | |
|---|---|---|---|---|---|---|---|
| | Test diet group | Placebo | Test diet group | Placebo | Test diet group | Placebo | |
| | (14 subject) | (15 subject) | (14 subject) | (15 subject) | (14 subject) | (15 subject) | Test |
| MoCA | 22.4 ± 2.1 | 22.3 ± 2.0 | 24.1 ± 1.5 | 23.1 ± 1.5 | 1.8 ± 2.2 | 0.7 ± 2.1 | NS |
| MMSE | 25.6 ± 2.3 | 26.2 ± 3.0 | 27.5 ± 1.7 | 26.3 ± 2.7 | 1.9 ± 2.0 | 0.1 ± 2.5 | P = 0.04 |
| CD result | 0 | 0 | 0 | 2 | | | |

### (1) Manufacture of Imidapeptide Compound

3000 L of water was added to 2000 kg of white salmon (semi-dressed) fillet, and the salmon extract obtained by heating and extracting at 80° C. for 30 minutes was filtered, adjusted to pH 5.0, passed through a cation exchange resin (Mitsubishi Chemical Corporation, DIAION SK-1B) column, and adsorbed on the cation exchange resin, and the anserine was eluted with 0.5% ammonia solution. The eluate was desalted and concentrated with an NF membrane (Daicen Membrane, Desal DL) to prepare an anserine solution from which creatinine and salts were removed.

Dextran was added to the eluate so that the anserine content was 30% and spray-dried to obtain about 40 kg of purified salmon anserine powder. Using the powder, 75 mg of vitamin C and 15 mg ferulic acid were added to 250 mg anserine content in the powder, and a citrus seasoning was further added to prepare approximately 5 kg of granules, which was divided into 2 g per one aluminum laminated bag to prepare 2500 packages.

The composition for improving mild cognitive impairment according to the present invention can be taken, for example, as a functional food.

What is claimed is:

1. A method for improving mild cognitive impairment of a human patient with mild cognitive impairment with a score of 23 points or more and less than 27 points in the Mini-Mental State Examination, a score of 0.5 in the Clinical Dementia Rating, or a score of less than 25 points in the Montreal Cognitive Assessment, the method comprising:
providing a dose of 250 mg-500 mg of a composition once or twice a day for three months or more,
the composition comprising imidazole dipeptide separated and purified from chicken extract or salmon extract, wherein the imidazole dipeptide does not contain creatinine, and 75% or more of the imidazole dipeptide in the composition is anserine.
